(19) United States
(12) Patent Application Publication
(10) Pub. No.: US 2025/0215475 A1
(43) Pub. Date: Jul. 3, 2025

(54) METHOD OF EVALUATING NEUTROPHIL ACTIVITY

(71) Applicant: HAMAMATSU PHOTONICS K.K., Hamamatsu-shi, (JP)

(72) Inventors: Kimiko KAZUMURA, Hamamatsu-shi (JP); Kozo TAKEUCHI, Hamamatsu-shi, (JP)

(21) Appl. No.: 19/051,880

(22) Filed: Feb. 12, 2025

Related U.S. Application Data
(62) Division of application No. 16/435,710, filed on Jun. 10, 2019.

(30) Foreign Application Priority Data
Jun. 11, 2018 (JP) 2018-111144

(51) Int. Cl. C12Q 1/28; G01N 21/76; G01N 33/50

(52) U.S. Cl. CPC C12Q 1/28; G01N 21/76; G01N 33/5094; G01N 2800/7009

(57) ABSTRACT

The present invention relates to a method of evaluating neutrophil activity, which includes: evaluating neutrophil activity based on an index derived from measurement results of myeloperoxidase (MPO) activity or superoxide production of a biological sample to which a neutrophil stimulant is added, in which the index is a time from addition of the neutrophil stimulant to rise of a peak of the MPO activity or the superoxide production.

### CROSS-REFERENCE TO RELATED APPLICATIONS

This application is a divisional application of U.S. application Ser. No. 16/435,710, filed Jun. 10, 2019, which claims the benefit of priority to Japanese Patent Application No. 2018-111144, filed Jun. 11, 2018, the entire contents of which are incorporated herein by reference.

### TECHNICAL FIELD

The present invention relates to a method of evaluating neutrophil activity.

### BACKGROUND

Neutrophils show migratory properties against inflammatory cytokines and bacteria, etc., thereby collecting in the inflamed part, having a function of killing bacteria etc. by phagocytosis, and playing an important role in biological defense. Production of reactive oxygen species (ROS; superoxide, hydrogen peroxide and the like) and production of hypochlorous acid by myeloperoxidase (MPO) are known as a mechanism of action by which neutrophils deactivate bacteria and the like.

On the other hand, it has been pointed out that the production of ROS by excessively activated neutrophils provokes oxidative stress, damage to host cells and tissues and thus is involved in various pathologies (refer to, for example, Journal of Analytical Bio-Science, Vol. 35, No. 2, 2012, pp. 133-139). In recent years, it has also been found that MPO released from neutrophils is also involved in lipid peroxidation, which is another source of oxidative stress (refer to, for example, Journal of Analytical Bio-Science, Vol. 35, No. 2, 2012, pp. 133-139).

As a method of evaluating the function of neutrophils, for example, Japanese Unexamined Patent Publication No. 2015-084757 discloses a method of assessing neutrophil activity based on measured MPO activity and superoxide production, which includes: a step of measuring MPO activity and superoxide production using the same sample containing whole blood, in which at least one of the MPO activity measurement and the superoxide production measurement is based on the fluorescence detection, which is carried out by irradiating a sample container containing the sample with excitation light output from an excitation light source and by detecting the emitted fluorescence with a fluorescence detector, and the excitation light source and the fluorescence detector are disposed on the same side with respect to an irradiated surface of the excitation light of the sample container.

### SUMMARY

A neutrophil takes bacteria and fungi so as to entrap them with neutrophil plasma membrane upon contact with those organisms, thereby forming a phagosome. The phagosome then fuses with granules and the granule contents are released into the phagosome. Reactive oxygen species (ROS; superoxide and hydrogen peroxide) is generated by NADPH oxidase system formed in a cell membrane (membrane of the phagosome) to sterilize bacteria and fungi. In addition, hypochlorous acid (HOCl) (or its halogen equivalents) is produced from hydrogen peroxide (H₂O₂) and chloride ion (Cl⁻) by the enzyme reaction of myeloperoxidase (MPO; EC No. 1.11.2.2) contained in the granule content, thereby sterilizing bacteria and fungi.

In the method disclosed in Japanese Unexamined Patent Publication No. 2015-084757, for example, MPO activity and superoxide production are derived by quantifying ROS and hypochlorous acid (or its halogen equivalents) after adding a neutrophil stimulant, and calculating a peak area. The neutrophil activity is evaluated on the basis of the MPO activity and the superoxide production.

Meanwhile, the inventors have found that, in a case of subjects who are thought to be in a situation exposed to oxidative stress state due to excessively activated neutrophils, caused by diseases such as arteriosclerosis and excessive fatigue, a peak takes a longer time to rise when the MPO activity and the superoxide production are measured by quantifying ROS and hypochlorous acid (or its halogen equivalents) after adding the neutrophil stimulant in accordance with the method as set forth in Japanese Unexamined Patent Publication No. 2015-084757. The present invention is made based on this newly found knowledge, of which an object is to provide a method of evaluating neutrophil activity with a new index.

The present invention provides a method of evaluating neutrophil activity, including: evaluating neutrophil activity based on an index derived from measurement results of MPO activity or superoxide production of a biological sample to which a neutrophil stimulant is added, in which the index is a time from addition of the neutrophil stimulant to rise of a peak of the MPO activity or the superoxide production.

The evaluation method according to the present invention takes the time from addition of the neutrophil stimulant to rise of the peak of the MPO activity or the superoxide production (hereinafter also referred to as “index time”) as an index, thus the neutrophil activity can be evaluated. The index time is longer for the subjects who are thought to be in a situation exposed to oxidative stress state due to excessively activated neutrophils, caused by diseases such as arteriosclerosis and excessive fatigue. The lengthen index time is considered to be triggered by that the neutrophil is excessively activated and thus its sensitivity to the next stimulus is reduced. Therefore, the evaluation method according to the present invention can also be regarded as, for example, a method of evaluating the neutrophil sensitivity.

The evaluating may include comparing the index time to a reference value and determining that neutrophil sensitivity is reduced in a case where the index time is greater than the reference value.

The biological sample is preferably a sample containing whole blood. Since the sample containing whole blood does not have to separate the neutrophils, the operation is not complicated, the neutrophils can be prevented from being stressed due to separation, and it is possible to obtain information closer to actual in vivo dynamics including interaction with various liquid factors contained in the blood.

The evaluating further may include evaluating the neutrophil activity based on a peak area of the MPO activity or the superoxide production in addition to the evaluating the neutrophil activity based on the index time. Sometimes decrease in a peak area is observed in the subject whose index time is longer. Therefore, the neutrophil activity can be multilaterally evaluated together with the evaluation based on the peak area. Additionally, based on the peak area, it is also possible to evaluate extrinsic factors such as “meal” and the anti-inflammatory ability (antioxidative ability or anti-oxidative stress ability) such as antioxidative enzymes contained in the living body.

According to the evaluation method of the present invention, it is possible to recognize subjects who are thought to be in a situation exposed to oxidative stress state due to excessively activated neutrophils, caused by diseases such as arteriosclerosis and excessive fatigue. Therefore, the present invention also provides a method of collecting data for determining whether or not a subject is suffering from a disease associated with oxidative stress due to excessively activated neutrophils, or whether or not the subject is in a condition associated with oxidative stress due to excessively activated neutrophils, which includes: deriving the data from measurement results of MPO activity or superoxide production of a biological sample to which a neutrophil stimulant is added, in which the data is a time from addition of the neutrophil stimulant to rise of a peak of the MPO activity or the superoxide production, and the biological sample is a biological sample collected from the subject.

According to another knowledge found by the inventors, it is considered that the index time is shorter in the subjects who are thought to be in a situation exposed to oxidative stress state due to excessively activated neutrophils, caused by diseases such as arteriosclerosis and excessive fatigue, when such a situation is improved. Therefore, the present invention further provides a method of collecting data for determining effect of treatment on a disease or a condition associated with oxidative stress due to excessively activated neutrophils, in a subject, which includes: deriving the data from measurement results of MPO activity or superoxide production of a biological sample to which a neutrophil stimulant is added, in which the data is a time from addition of the neutrophil stimulant to rise of a peak of the MPO activity or the superoxide production, and the biological sample is a biological sample collected from the subject after the treatment.

According to the present invention, it is possible to evaluate the neutrophil activity with a new index. The evaluation method of the present invention is also capable of evaluating the neutrophil sensitivity since the evaluation is carried out with the index time as an index. Moreover, according to the present invention, it is possible to determine whether or not the subject is suffering from a disease associated with oxidative stress due to excessively activated neutrophils or whether or not the subject is in a condition associated with oxidative stress due to excessively activated neutrophils, or alternatively, to determine the effect of treatment on the disease or the condition associated with oxidative stress due to excessively activated neutrophils in the subject.

### BRIEF DESCRIPTION OF THE DRAWINGS

FIG. 1A is a graph illustrating measurement results of myeloperoxidase (MPO) activity (fluorescence intensity) and superoxide production (chemiluminescence intensity) in Test Example 1, which are measured using biological samples containing whole blood collected from a patient (patient number: 003) suffering from atherosclerosis obliterans (ASO) of lower extremity and being subjected to peripheral vascular catheterization treatment.

FIG. 1B is a graph illustrating measurement results of MPO activity and superoxide production in Test Example 1, which are measured using biological samples containing whole blood collected from a patient (patient number: 009) suffering from ASO of lower extremity and being subjected to peripheral vascular catheterization treatment.

FIG. 2A is a graph illustrating a result of analysis of a correlation between a peak area of superoxide (O₂^{-⋅}) production and a peak area of MPO activity, before treatment, in Test Example 1.

FIG. 2B is a graph illustrating a result of analysis of a correlation between an index time of superoxide (O₂^{-⋅}) production and a peak area of MPO activity, before treatment, in Test Example 1.

FIG. 2C is a graph illustrating a result of analysis of a correlation between an index time of superoxide (O₂^{-⋅}) production and a peak area of O₂^{-⋅} production, before treatment, in Test Example 1.

FIG. 2D is a graph illustrating a result of analysis of a correlation between a peak area of superoxide (O₂^{-⋅}) production and a peak area of MPO activity, one month after treatment, in Test Example 1.

FIG. 2E is a graph illustrating a result of analysis of a correlation between an index time of superoxide (O₂^{-⋅}) production and a peak area of MPO activity, one month after treatment, in Test Example 1.

FIG. 2F is a graph illustrating a result of analysis of a correlation between an index time of superoxide (O₂^{-⋅}) production and a peak area of O₂^{-⋅} production, one month after treatment, in Test Example 1.

FIG. 3 is a graph illustrating measurement results of MPO activity (fluorescence intensity) and superoxide production (chemiluminescence intensity) using a biological sample containing whole blood collected from a healthy individual in Test Example 2.

FIG. 4A is a graph illustrating results of deriving index times from measurement results of superoxide (O₂^{-⋅}) production using biological samples containing whole blood collected from healthy individuals in Test Example 3.

FIG. 4B is a graph illustrating results of deriving index times from measurement results of MPO activity using biological samples containing whole blood collected from healthy individuals in Test Example 3.

FIG. 5A is a graph illustrating results of deriving index times from measurement results of superoxide (O₂^{-⋅}) production using biological samples containing whole blood collected from patients suffering from ASO of lower extremity (before treatment) in Test Example 3.

FIG. 5B is a graph illustrating results of deriving index times from measurement results of MPO activity using biological samples containing whole blood collected from patients suffering from ASO of lower extremity (before treatment) in Test Example 3.

FIG. 6A is a graph illustrating results of deriving index times from measurement results of superoxide (O₂^{-⋅}) production using biological samples containing whole blood collected from patients suffering from ASO of lower extremity (one month after catheterization treatment) in Test Example 3.

FIG. 6B is a graph illustrating results of deriving index times from measurement results of MPO activity using biological samples containing whole blood collected from patients suffering from ASO of lower extremity (one month after catheterization treatment) in Test Example 3.

FIG. 7A is a graph illustrating results of deriving index times from measurement results of superoxide (O₂^{-⋅}) production using biological samples containing whole blood collected from patients suffering from brain disease in Test Example 3.

FIG. 7B is a graph illustrating results of deriving index times from measurement results of MPO activity using biological samples containing whole blood collected from patients suffering from brain disease in Test Example 3.

FIG. 8A is a graph illustrating a result of analysis of a correlation between MPO activity in plasma and MPO activity during stimulation (i.e. MPO activity measured after adding a neutrophil stimulant) using the biological sample (before treatment) of Test Example 1, in Reference Example.

FIG. 8B is a graph illustrating a result of analysis of a correlation between MPO activity in plasma and MPO activity during stimulation (i.e. MPO activity measured after adding a neutrophil stimulant) using the biological sample (one month after treatment) of Test Example 1, in Reference Example.

### DETAILED DESCRIPTION

Hereinafter, modes for carrying out the present invention will be described in detail. However, the present invention is not limited to the following embodiments.

A method of evaluating neutrophil activity according to the present embodiment (hereinafter also referred to as an “evaluation method”) includes a step of evaluating (hereinafter also referred to as an “evaluation step”) neutrophil activity based on an index derived from measurement results of myeloperoxidase (MPO) activity or superoxide production of a biological sample to which a neutrophil stimulant is added, in which the index is a time from addition of the neutrophil stimulant to rise of a peak of the MPO activity or the superoxide production.

The evaluation method according to the present embodiment may further include a step of adding (hereinafter also referred to as an “addition step”) a neutrophil stimulant to the biological sample, a step of measuring (hereinafter also referred to as a “measurement step”) the MPO activity or the superoxide production using the biological sample to which the neutrophil stimulant is added, and a step of deriving (hereinafter also referred to as a “derivation step”) the index (index time) from the measurement results of the MPO activity or the superoxide production. However, the evaluation method according to the present embodiment does not necessarily have to include the addition step, the measurement step and the derivation step, but may be configured to carry out the evaluation step only using the index time separately derived.

The biological sample may be a sample containing neutrophils collected from a living body. Neutrophils are contained in, for example, blood, saliva, and gingival crevicular fluid. The biological sample may be, for example, a sample collected from the living body (e.g. blood (whole blood), saliva, and gingival crevicular fluid), or may be a sample containing a treated fluid obtained by processing the sample collected from the living body in which the neutrophils are concentrated or separated. The treated fluid in which the neutrophils are concentrated or separated can be prepared according to conventional methods such as, for example, a method of concentrating or separating the neutrophils using flow cytometry.

The biological sample may be a sample collected from the living body or a treated fluid itself, or may be a solution obtained by diluting the sample collected from the living body or the treated fluid with physiological saline, buffer solution or the like. In a case of dilution, a dilution ratio may be set appropriately to the extent that the MPO activity or the superoxide production can be detected. For example, when whole blood is diluted, it is preferable to dilute 10 to 750 times, more preferably 50 to 500 times, still more preferably 100 to 400 times, further still more preferably 200 to 300 times, and particularly preferably 220 to 280 times.

The neutrophil stimulant may be any substance that activates neutrophil function (e.g. migration or phagocytosis). Examples of the neutrophil stimulant include formyl methionyl leucyl phenylalanine (fMLP: neutrophil chemotactic peptide), phorbol 12-myristate 13-acetate (PMA) and opsonized zymon (OZ), which may be used alone or in combination of two or more.

The MPO activity or the superoxide production can be measured, for example, by detecting fluorescence, luminescence or absorbance over time using a fluorescent indicator, a luminescent (e.g. chemiluminescent) indicator or a light-absorbing indicator. The indicator may be a commercially available reagent, or may be a commercially available measurement kit.

The MPO activity can be measured, for example, using a fluorescent indicator, a luminescent indicator or a light-absorbing indicator, which reacts with hypochlorous acid (or its halogen equivalents) produced by MPO. Examples of such an indicator include aminophenyl fluorescein (APF), taurine/TNB (refer to J. Clin. Invest., Vol. pp. 70, 598-607, 1982), and 8-amino-5-chloro-7-phenylpyrido[3,4-d]pyridazine-1,4-(2H,3H) dione (L-012: refer to Anal Biochem., Vol. 271 (1), pp. 53-58, 1999).

The reaction between APF and HOCl can be detected by fluorescence (for example, at an excitation wavelength of 490 nm and at a fluorescence wavelength of 515 nm). The reaction between taurine/TNB and HOCl can be detected by absorbance (for example, at a wavelength of 412 nm). The reaction between L-012 and HOCl can be detected, although less specific, by chemiluminescence (for example, at a wavelength of 455 nm).

The measurement of superoxide production can be measured, for example, using a fluorescent indicator, a luminescent indicator or a light-absorbing indicator, which reacts with superoxide. Examples of such an indicator include 2-methyl-6-phenyl-3,7-dihydroimidazo[1,2-a]pyrazin-3-one (CLA), 2-methyl-6-(4-methoxyphenyl)-3,7-dihydroimidazo[1,2-a]pyrazin-3-one (MCLA), 2-methyl-6-p-methoxyphenylethynylimidazopyrazinone (MPEC), indocyanine-type imidazopyranodine compound (NIR-CLA), and 2-[2,4,5,7-tetrafluoro-6-(2-nitro-4,5-dimethoxyphenylsulfonyloxy)-3-ox o-3H-xanthen-9-yl]benzoic acid (BES-So).

The reaction between CLA and superoxide can be detected by chemiluminescence (for example, at a maximum emission wavelength of 380 nm). The reaction between MCLA and superoxide can be detected by chemiluminescence (for example, at a maximum emission wavelength of 465 nm). The reaction between MPEC and superoxide can be detected by chemiluminescence (for example, at a maximum emission wavelength of 430 nm). The reaction between NIR-CLA and superoxide can be detected by chemiluminescence (for example, at a maximum emission wavelength of 800 nm). The reaction between BES-So and superoxide can be detected by fluorescence (for example, at an excitation wavelength of 505 nm and at a fluorescence wavelength of 544 nm).

Evaluation by the evaluation method according to the present embodiment can be carried out if only one of the MPO activity and the superoxide production is measured. However, both MPO activity and superoxide production may be measured from the same biological sample according to, for example, the method described in Japanese Unexamined Patent Publication No. 2015-084757.

The detection of fluorescence, luminescence or absorbance can be carried out using known devices such as, for example, a fluorometer, a luminescence measuring device, an absorptiometer and the like. Moreover, it is preferable to carry out the fluorescence detection according to the method described in Japanese Unexamined Patent Publication No. 2015-084757 in a case where biological sample contains whole blood.

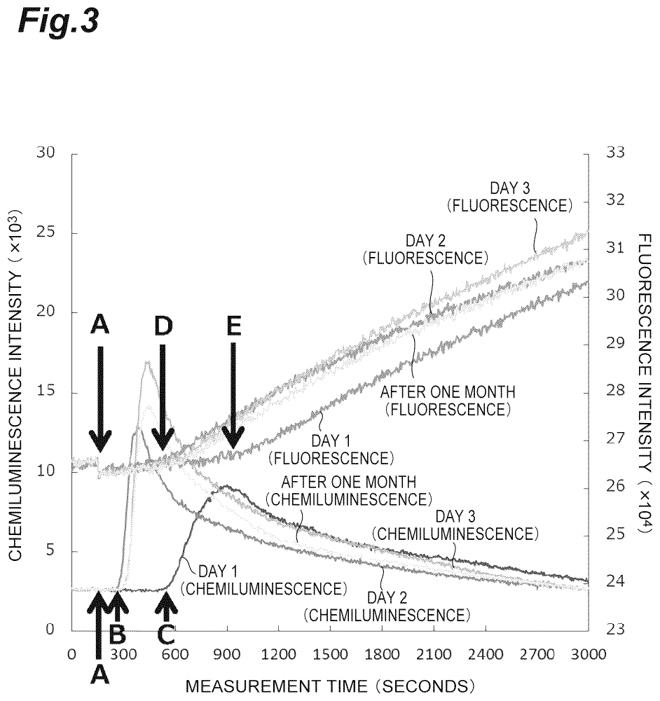

The index time is a time from a point at which the neutrophil stimulant is added to a point at which the peak of MPO activity or superoxide production rises. The derivation method of the index time will be described with reference to FIG. 3. FIG. 3 is a graph illustrating measurement results of the MPO activity and the superoxide production using a biological sample containing whole blood collected from a healthy individual. In the graph of FIG. 3, a horizontal axis indicates elapsed time (measurement time), and a right vertical axis and a left vertical axis indicate the MPO activity (fluorescence intensity (measurement value)) the and superoxide production (chemiluminescence intensity) (measurement value)), respectively.

The graph of FIG. 3 illustrates measurement results obtained and summarized by as follows: 1) the MPO activity and the superoxide production are measured on respective biological samples (samples containing whole blood) for which blood is collected every morning for three consecutive days from the same healthy individual; and 2) the MPO activity and the superoxide production are measured on biological samples (samples containing whole blood) obtained by collecting blood again about one month later. Additionally, the healthy individual complained on day 1 that she still felt extreme tired because of overworks until the day before, which is thought to correspond to a situation exposed to oxidative stress due to excessively activated neutrophils.

Arrow A in FIG. 3 indicates a point at which the neutrophil stimulant is added. Arrows B and C indicate points at which the peak of the superoxide production rises, as measured using biological samples collected on day 2 and day 1, respectively. Similarly, arrows D and E indicate points at which the peak of the MPO activity rises, as measured using the biological samples collected on day 2 and day 1, respectively. For example, a time from the point indicated by arrow A to the point indicated by arrow C is an index time derived from the superoxide production measured in the biological sample collected on day 1. Moreover, for example, a time from the point indicated by arrow A to the point indicated by arrow B is an index time derived from the superoxide production measured in the biological sample collected on day 2. Additionally, in a case of an example shown in FIG. 3, an index time on day 1 when the subject (healthy individual) complains of extreme fatigue shows a larger value than the index time on day 2. As can be understood from FIG. 3, if either MPO activity or superoxide production is measured, it is possible to evaluate using the evaluation method according to the present embodiment.

The time when the peak of the MPO activity or the superoxide production rises can be identified as, for example, a point (near a tail of the peak) when a slope of the measurement value starts to change relative to the background level from a graph in which the measurement value is plotted with respect to the measurement time, after the neutrophil stimulant is added to the biological sample and the measurement is continued until the peak of the MPO activity or the superoxide production appears.

In a case where the MPO activity is measured by, for example, quantifying hypochlorous acid (or its halogen equivalents) produced by MPO, the index time usually falls within a range of 290 to 470 seconds. This index time changes to be longer (e.g. longer than 500 seconds), for example, in a subject who is thought to be in a situation exposed to oxidative stress state due to excessively activated neutrophils, caused by diseases such as arteriosclerosis and excessive fatigue, since sensitivity to the next stimulus is reduced in the neutrophils.

In a case where the superoxide production is measured by, for example, quantifying superoxide, the index time usually falls within a range of 65 to 150 seconds. This index time changes to be longer (e.g. longer than 200 seconds), for example, in a subject who is thought to be in a situation exposed to oxidative stress state due to excessively activated neutrophils, caused by diseases such as arteriosclerosis and excessive fatigue, since sensitivity to the next stimulus is reduced in the neutrophils.

The neutrophil activity can be evaluated based on the index time derived. As described above, when the index time is a larger value, it means that the sensitivity to stimulation is reduced in the neutrophils. For example, the neutrophil sensitivity (sensitivity to stimulation) may be evaluated as reduced when the derived index time exceeds a certain predetermined reference value. Similarly, the neutrophil sensitivity (sensitivity to stimulation) may be evaluated as normal when the derived index time is near a predetermined reference value.

The reference value may be appropriately set in accordance with a purpose. The reference value may be derived by, for example, averaging index times measured from a lot of human samples. A plurality of reference values may be set in accordance with, for example, sex, age, and the like. Also, for a specific individual, a value measured in a healthy state may be used as the reference value. Furthermore, in the method of collecting data for determining the effect of a treatment on the disease or the condition associated with oxidative stress due to excessively activated neutrophils, the index time before treatment can also be used as the reference value.

The evaluation method according to the present embodiment may further include, in the evaluation step, evaluating the neutrophil activity based on a peak area of the MPO activity or the superoxide production in addition to the evaluating the neutrophil activity based on the index time. The neutrophil activity can be multilaterally evaluated by carrying out the evaluation based on the peak area. Additionally, based on the peak area, it is also possible to evaluate extrinsic factors such as “meal” and the anti-inflammatory ability (antioxidative ability or anti-oxidative stress ability) such as antioxidative enzymes contained in the living body.

For example, the evaluation method according to the present embodiment can be applied to a method of collecting data (first data collection method) for determining whether or not the subject is suffering from a disease associated with oxidative stress due to excessively activated neutrophils, or whether or not the subject is in a condition associated with oxidative stress due to excessively activated neutrophils, and also applied to a method of collecting data (second data collection method) for determining effect of treatment on a disease or a condition associated with oxidative stress due to excessively activated neutrophils in the subject.

The first data collection method according to the present embodiment includes a step of deriving an index time from the measurement results of the MPO activity or the superoxide production of the biological sample to which the neutrophil stimulant is added, in which the derived index time is collected as the data. The biological sample is a biological sample obtained from a subject.

Examples of the diseases associated with oxidative stress due to excessively activated neutrophils include arteriosclerosis such as atherosclerosis obliterans (ASO) of lower extremity, cancer, inflammatory diseases, hyperlipidemia, diabetes, Alzheimer's disease, Parkinson's disease, alcoholism, and the like. Examples of the condition associated with oxidative stress due to excessively activated neutrophils include excessive fatigue, post-intensive exercise state, smoking, aging, and the like.

The second data collection method according to the present embodiment includes a step of deriving an index time from the measurement results of the MPO activity or the superoxide production of the biological sample to which the neutrophil stimulant is added, in which the derived index time is collected as the data. The biological sample is a biological sample obtained from a subject after treatment.

The second data collection method may further include a step of deriving the index time for the biological sample obtained from a subject before treatment and further collecting the derived index time (before treatment) as the data.

Specific embodiments of the first and second data collection methods according to the present embodiment are as described above.

### EXAMPLES

Hereinafter, the present invention will be more specifically described based on examples. However, the present invention is not limited to the following examples.

### Test Method

In the example, the evaluation of the neutrophil activity was performed in accordance with the following method.

### Measurement of Myeloperoxidase Activity and Superoxide Production

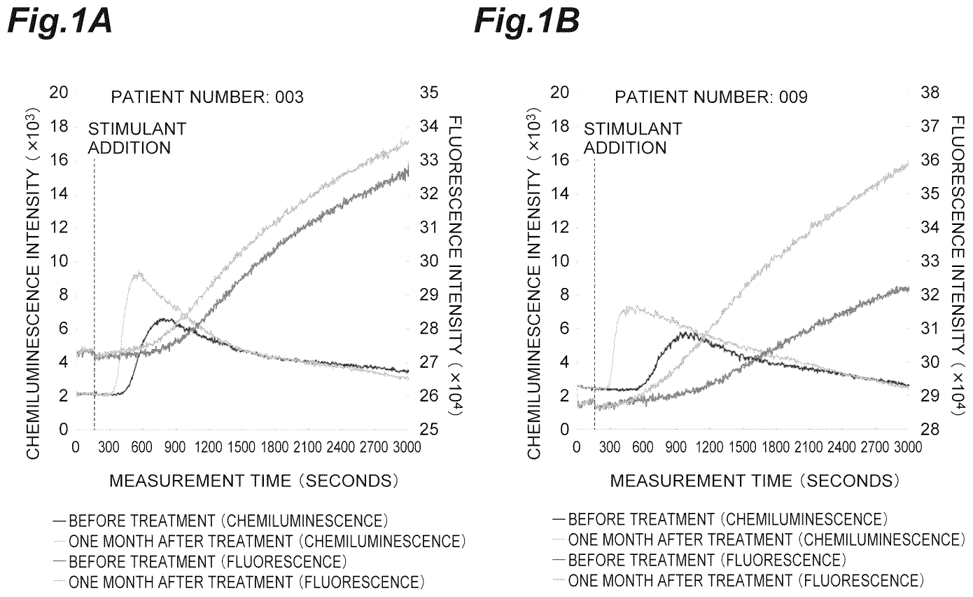

The myeloperoxidase (MPO) activity and the superoxide production were measured according to the method described in the example of Japanese Unexamined Patent Publication No. 2015-084757. In particular, a container equivalent to a measurement container described in FIG. 1 of Japanese Unexamined Patent Publication No. 2018-025480 was used as a sample container. MCLA (trade name, manufactured by Tokyo Chemical Industry Co., Ltd.) represented by the following formula (1), APF (trade name, manufactured by Sekisui Medical Co., Ltd.) represented by the following formula (2), and CaCl₂ were added to RH buffer (10 mM HEPES, 154 mM NaCl, 5.6 mM KCl, pH 7.4), which was kept at 37° C., so as to concentrations of MCLA, APF and CaCl₂ were 0.5 μM, 10 μM and 1 mM, respectively. The resultant mixture was incubated at 37° C. for 4 minutes. 3 μL of blood (whole blood) collected from a subject was added to the incubated solution (250-fold dilution) and the resultant solution was used as a biological sample. The biological sample was incubated at 37° C. for 1 minute, and then was measured using a fluorescence and luminescence simultaneous measurement device.

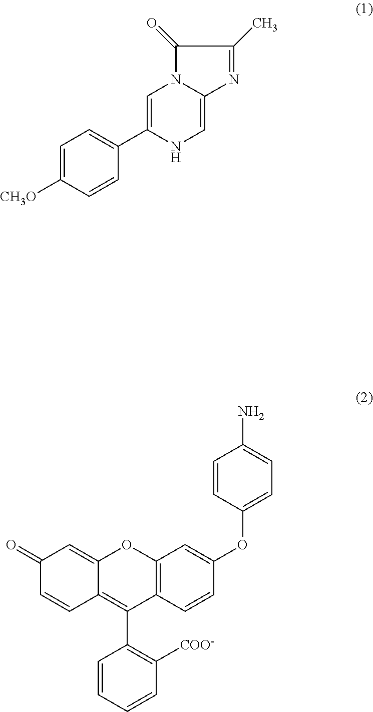

The biological sample was irradiated with a LED (480 nm) blinking at intervals of 125 ms, as excitation light. After 2.5 minutes, a protein kinase C activator (PMA (phorbol 12-myristate 13-acetate): neutrophil stimulant) was added to the biological sample so as to concentration of PMA was 0.1 μM. APF fluorescence intensity (myeloperoxidase activity) and MCLA chemiluminescence intensity (superoxide production) were measured.

### Derivation of Index Time

The index time was derived from a graph in which the APF fluorescence intensity and the MCLA chemiluminescence intensity, measured by the method described above, were plotted with respect to a time (measurement time). The index time is a time from when the neutrophil stimulant is added (stimulant addition) to when the peak of the MPO activity or the superoxide production rises. The point at which the peak of the MPO activity or the superoxide production rises is identified as a point at which a slope of the measurement value of fluorescence intensity or chemiluminescence intensity changes relative to the background level (near the tail of the peak).

### Test Example 1

Evaluation for Patients Suffered from Arteriosclerosis

Blood samples were collected from the subjects (patients suffering from atherosclerosis obliterans (ASO) of lower extremity and being subjected to peripheral vascular catheterization treatment) before and one month after the catheterization treatment. The MPO activity (fluorescence intensity) and the superoxide production (chemiluminescence intensity) were measured by the test method described above using whole blood collected. The results for representative subjects (two patients) are shown in FIG. 1A and FIG. 1B.

As shown in FIG. 1A and FIG. 1B, the points at which the peaks of the MPO activity and the superoxide production rise one month after the treatment for both patients are earlier than those before treatment (that is, the index time is reduced). In both patients, high effect of the catheterization treatment was achieved, symptoms were alleviated, and the ABI value (Ankle Brachial Pressure Index), which is a conventional index for evaluating stenosis and occlusion of lower extremity artery, was also improved. The index times derived from the graphs of FIG. 1A and FIG. 1B are as summarized in Table 1 below.

TABLE 1

| Subject | | | Index Time |
|---|---|---|---|
| (Patient Number) | | | (seconds) |
| 003 | MPO Activity | Before | 589 |
| | | Treatment | |
| | | 1-month after | 523.5 |
| | | Treatment | |
| | Superoxide Production | Before | 250.5 |
| | | Treatment | |
| | | 1-month after | 160.5 |
| | | Treatment | |
| 009 | MPO Activity | Before | 810 |
| | | Treatment | |
| | | 1-month after | 214 |
| | | Treatment | |
| | Superoxide Production | Before | 326 |
| | | Treatment | |
| | | 1-month after | 120.5 |
| | | Treatment | |

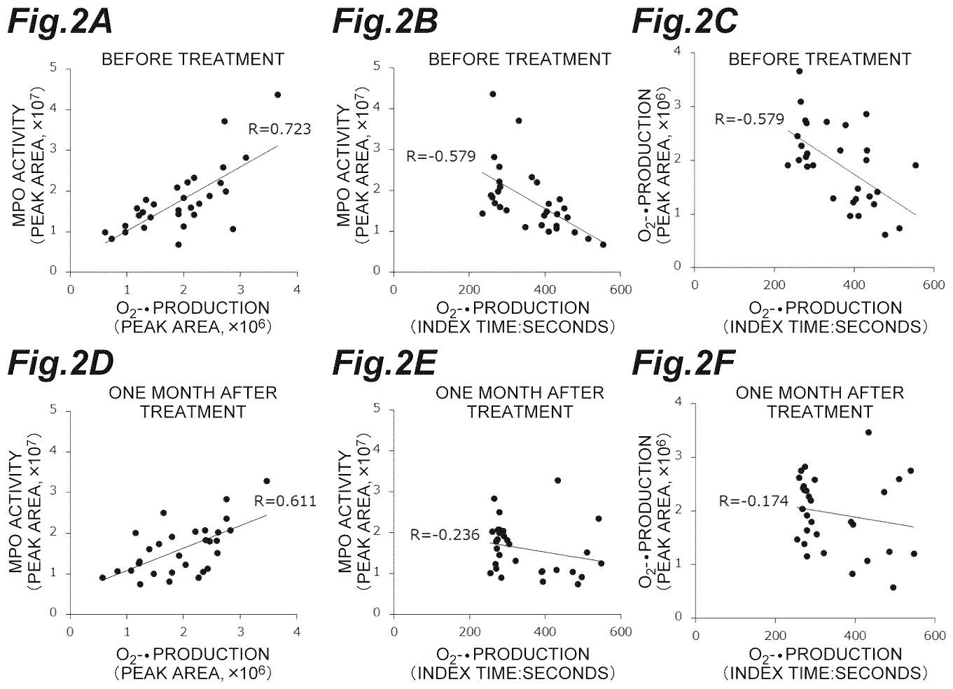

FIGS. 2A to 2F show results of analysis of correlations, using the data obtained from 30 patients (subjects), between a peak area of the MPO activity and a peak area of the superoxide (O₂^{-⋅}) production (FIG. 2A: before treatment, FIG. 2D: one month after treatment), between the peak area of the MPO activity and the index time of the O₂^{-⋅} production (FIG. 2B: before treatment, FIG. 2E: one month after treatment), and between the peak area of the O₂^{-⋅} production and the index time of the O₂^{-⋅} production (FIG. 2C: before treatment, FIG. 2F: one month after treatment). A correlation coefficient between the peak area of the O₂^{-⋅} production and the index time of the O₂^{-⋅} production was −0.579 before treatment, which showed a negative correlation (FIG. 2C). A correlation coefficient between the peak area of the MPO activity and the index time of O₂^{-⋅} production was the same value before treatment and showed a negative correlation (FIG. 2B). Additionally, the correlation decreased in both patients one month after treatment (FIG. 2E and FIG. 2F). In a case of a healthy individual, the index time is almost constant, and it is considered that the index time has no negative correlation with both peak areas. Therefore, the correlation is considered to be reduced due to the effect of the treatment.

### Test Example 2

### Evaluation for Healthy Individual

A healthy individual was chosen as a subject, from whom the blood samples were collected randomly (several times a week) (totally about 80 times). The MPO activity (fluorescence intensity) and the superoxide production (chemiluminescence intensity) were measured by the test method described above using whole blood collected. FIG. 3 is a graph illustrating measurement results of the same subject (healthy individual) for three consecutive days (day 1 to day 3 in FIG. 3) and after one month. The subject complained on day 1 that she still felt extreme tired because of overworks until the day before, which is thought to correspond to a situation exposed to oxidative stress state due to excessively activated neutrophils.

As shown in FIG. 3, according to the measurement results on the day on which the subject complained extreme fatigue, the peaks of the MPO activity (fluorescence intensity) and the superoxide production (chemiluminescence intensity) rose slower than usual. Among about 80 measurement results in total, points at which the peaks of the MPO activity and the superoxide production rose were delayed on this day only. Moreover, as for both activities, the peak areas decreased compared with usual. The peak area is usually about the same as the measurement result after about one month. The slightly larger peak area on day 3 is considered to be due to light exercise on the night of day 1. The peak area tends to increase gradually two days after exercise (day 3 in FIG. 3) due to the influence of light exercise and the like. The index times and peak areas derived from the graph of FIG. 3 are as summarized in Table 2 below.

TABLE 2

| | | | Index | | |
|---|---|---|---|---|---|
| | | | Time | | |
| Subject | | | (seconds) | Peak Area | Remarks |
| Healthy | MPO | Day 1 | 627.5 | 8524987 | complaining |
| Indi- | Activity | | | | extreme |
| vidual | | | | | fatigue; light |
| | | | | | exercise at |
| | | | | | night |
| | | Day 2 | 271.5 | 12603260 | — |
| | | Day 3 | 320.5 | 13265020 | — |
| | | After One | 347 | 11870905 | — |
| | | Month | | | |
| | Superoxide | Day 1 | 354.5 | 1415548 | complaining |
| | Production | | | | extreme |
| | | | | | fatigue; light |
| | | | | | exercise at |
| | | | | | night |
| | | Day 2 | 107 | 1346020 | — |
| | | Day 3 | 106 | 2049328 | — |
| | | After One | 132 | 1658331 | — |
| | | Month | | | |

### Test Example 3

### Evaluation for Patients and Healthy Individuals

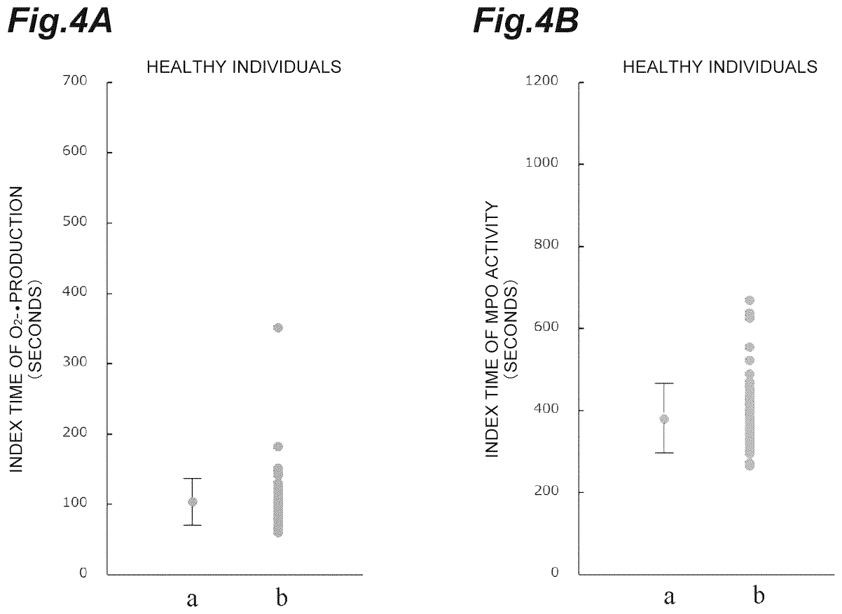

The MPO activity and superoxide (O₂^{-⋅}) production were measured using the test method described above for the subjects including healthy individuals, patients suffering from arteriosclerosis, and patients suffering from brain disease, thereby deriving the index times. The results are shown in FIGS. 4A to 7B and Table 3.

FIGS. 4A and 4B are graphs respectively illustrating index times derived from measurement results of the superoxide (O₂^{-⋅}) production and the MPO activity using biological samples containing whole blood collected from healthy individuals. The subjects were 13 healthy volunteers aged 20 to 50, each of whose whole blood was randomly collected to be used for measurement (76 samples in total). In FIG. 4A, “a” is obtained by plotting mean value and standard deviation of the index times derived from the O₂^{-⋅} production, and “b” is obtained by plotting the index times derived from the O₂^{-⋅} production. In FIG. 4B, “a” is obtained by plotting mean value and standard deviation of the index times derived from the MPO activity, and “b” is obtained by plotting the index times derived from the MPO activity. In healthy individuals, the index time derived from the O₂^{-⋅} production falls within a range of about 65 to 150 seconds, and the index time derived from the MPO activity falls within a range of about 290 to 470 seconds (FIGS. 4A and 4B, and Table 3).

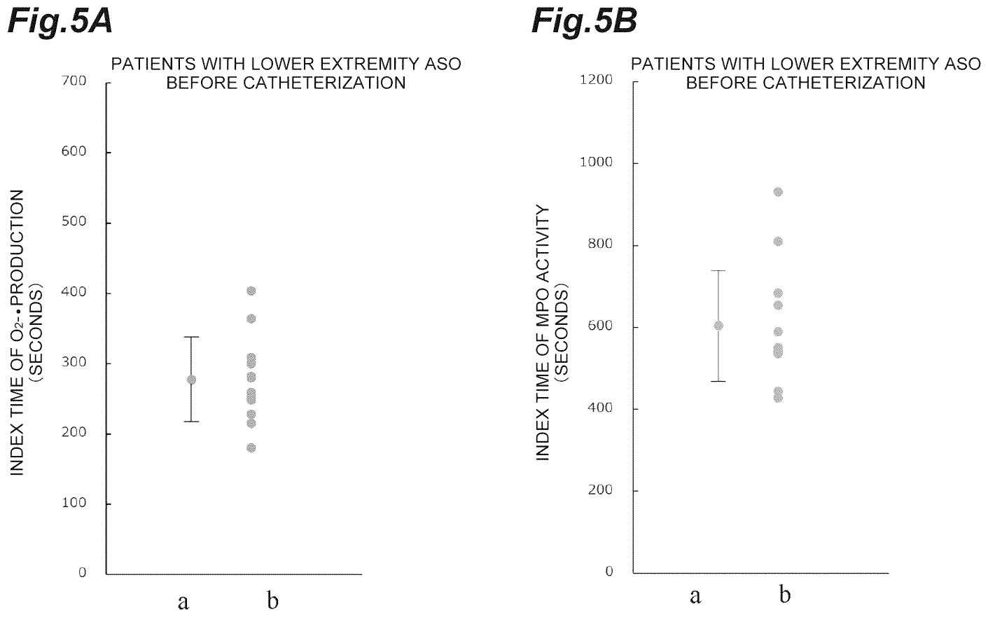

FIGS. 5A and 5B are graphs respectively illustrating index times derived from measurement results of the superoxide (O₂^{-⋅}) production and the MPO activity using biological samples containing whole blood collected from patients suffering from ASO of lower extremity (before treatment). The subjects were 12 patients suffering from ASO of lower extremity, each of whose index time exceeded the range measured in the healthy individuals (described above) before treatment and the effect of catheterization treatment was observed. In FIGS. 5A and 5B, “a” and “b” are the same as in FIGS. 4A and 4B (that is, “a” is obtained by plotting mean value and standard deviation of the index times, and “b” is obtained by plotting the index times).

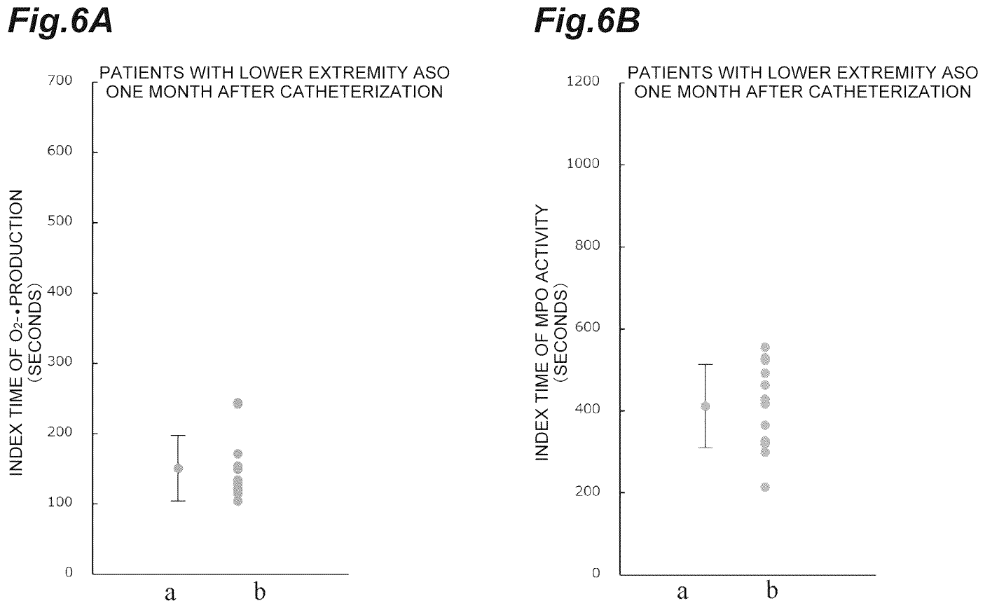

FIGS. 6A and 6B are graphs respectively illustrating index times derived based on measurement results of the superoxide (O₂^{-⋅}) production and the MPO activity using biological samples containing whole blood collected from patients suffering from ASO of lower extremity (one month after treatment). The subjects were the same as FIGS. 5A and 5B. In FIGS. 6A and 6B, “a” and “b” are the same as in FIGS. 4A and 4B (that is, “a” is obtained by plotting mean value and standard deviation of the index times, and “b” is obtained by plotting the index times). As shown in FIGS. 5A to 6B and Table 3, both the index times derived from the MPO activity and the O₂^{-⋅} production were reduced after catheterization treatment. On the other hand, the index time after catheterization treatment was larger than the index time observed in the healthy individual. It is considered that this phenomenon is due to that most of the subjects (patients) were elderly peoples and affected by aging, and that they had multiple diseases in addition to arteriosclerosis.

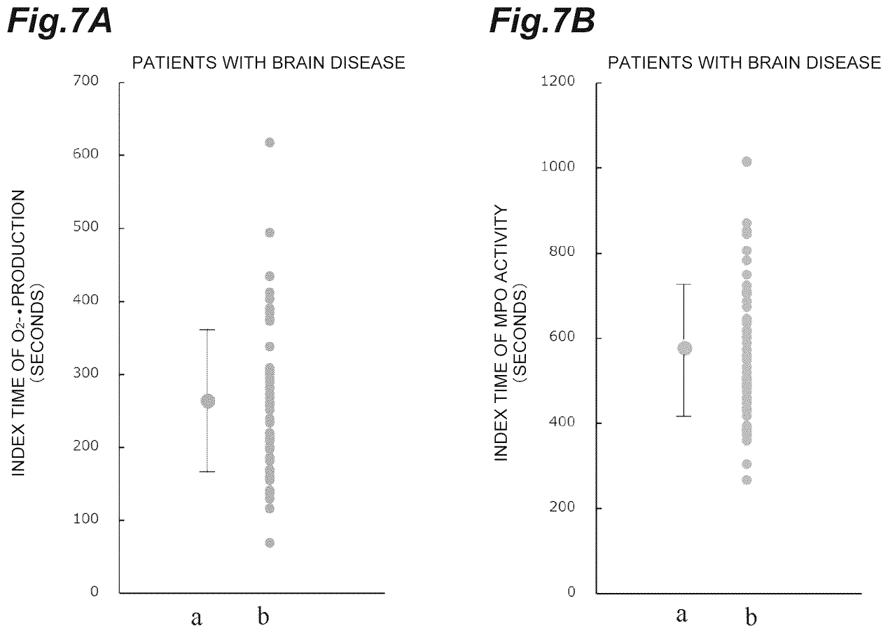

FIGS. 7A and 7B are graphs respectively illustrating index times derived based on measurement results of the superoxide (O₂^{-⋅}) production and the MPO activity using biological samples containing whole blood collected from patients suffering from brain disease. The subjects were 39 patients suffering from brain disease, each of whose whole blood was randomly collected to be used for measurement (48 samples in total). The subjects included 14 patients suffering from myasthenia gravis (16 samples in total), 11 patients suffering from multiple sclerosis (12 samples in total), 6 patients suffering from neuromyelitis optica (8 samples in total), 3 patients suffering from chronic inflammatory demyelinating polyneuritis (6 samples in total), 2 patients suffering from Miller-Fisher syndrome (3 samples in total), 1 patient suffering from Fabry disease (2 samples in total), and 1 patients suffering from Sweet syndrome (1 sample in total). In FIGS. 7A and 7B, “a” and “b” are the same as in FIGS. 4A and 4B (that is, “a” is obtained by plotting mean value and standard deviation of the index times, and “b” is obtained by plotting the index times). As shown in FIGS. 7A and 7B, and Table 3, the patients with brain disease have wide range of diseases and multiple stages, so that the derived index times have a large variation. Myasthenia gravis is an autoimmune disorder based on chronic inflammation. In many cases, the index time derived from the patients with brain disease greatly exceeds the range of the index time observed in the healthy individuals, thus it is assumed that those patients are in a condition associated with oxidative stress due to excessively activated neutrophils.

TABLE 3

| | | Superoxide | | | | |
|---|---|---|---|---|---|---|
| | | Production | | MPO Activity | | |
| | | Index Time | | Index Time | | |
| | | (seconds) | | (seconds) | | |
| | | Mean | Standard | Mean | Standard | Subjects |
| | | Value | Deviation | Value | Deviation | (Samples) |
| Healthy Individual | | 103.7 | 36.5 | 379.8 | 82.8 | 13 (76) |
| Patient | Before | 277.0 | 59.4 | 604.3 | 139.9 | 12 (12) |
| with ASO | Treatment | | | | | |
| of Lower | 1-month | 150.5 | 45.2 | 411.5 | 102.3 | 12 (12) |
| Extremity | after | | | | | |
| | Treatment | | | | | |

| | | | | | |
|---|---|---|---|---|---|
| Patient with Brain | 263.9 | 107.0 | 577.0 | 168.3 | 39 (48) |
| Disease | | | | | |

From the results shown in FIGS. 4A to 7B and Table 3, for example, in a case where the index time derived from the superoxide (O₂^{-⋅}) production (chemiluminescence intensity) exceeds 150 seconds, or the index time derived from the MPO activity (fluorescence intensity) exceeds 500 seconds, it is considered that the patient is suffering from a disease associated with oxidative stress due to excessively activated neutrophils, or is in a condition associated with oxidative stress due to excessively activated neutrophils. That is, for example, a reference value adopted for the index time derived from the O₂^{-⋅} production (chemiluminescence intensity) falls within a range of 150 to 200 seconds (e.g. 150 seconds or 200 seconds), and a reference value adopted for the index time derived from the MPO activity (fluorescence intensity) falls within a range of 500 to 550 seconds (e.g. 500 seconds or 550 seconds).

### Reference Example

Correlation between Index Time and with or without of Stimulation Caused by Neutrophil Stimulant

The MPO activity was measured using the whole blood collected from the subject of Test Example 1 according to the same procedure as the test method described above, and the peak area was calculated (MPO activity during stimulation). Plasma was separated from the same whole blood, and the MPO activity was measured (MPO activity in plasma) using a colorimetric assay kit (Ab105136, manufactured by Abcam). The MPO activity in plasma is activity of MPO secreted from neutrophils without the neutrophil stimulant acting on the neutrophils, and reflects the neutrophil activity in the subject at the time of collecting blood.

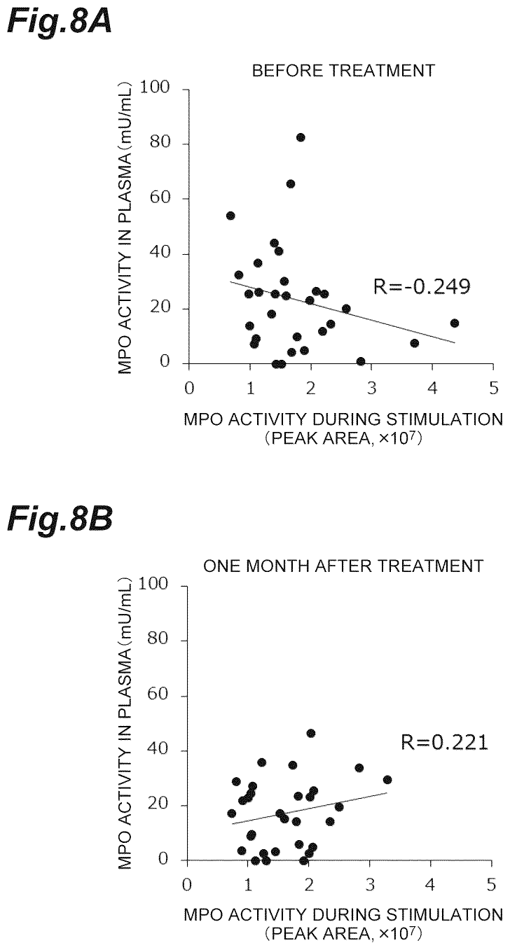

FIGS. 8A and 8B are graphs respectively illustrating results of analysis of a correlation between MPO activity in plasma (mU/mL) and MPO activity during stimulation (peak area, ×10⁷). FIG. 8A shows a result of analysis of the correlation before treatment, and FIG. 8B shows a result of analysis of the correlation one month after catheterization treatment. As shown in FIGS. 8A and 8B, a negative correlation was shown before treatment, while a positive correlation was shown one month after treatment. These results indicate that MPO activity is high and neutrophils are overactivated in the patients before treatment even without stimulation. Further the results indicate that the responsiveness (sensitivity) to the neutrophil stimulant is impaired in the patients before treatment. Thus it is assumed that the patients were in a condition associated with oxidative stress due to excessively activated neutrophils.

The peak area of the MPO activity or the superoxide production usually reflects the neutrophil activity. In a case where the peak area is small, the neutrophil activity is stable (not in oxidative stress state). In a case where the peak area is extremely small, it is considered that innate immune activity is reduced. On the other hand, based on the results shown in FIGS. 8A and 8B, even in a case where the peak area is small but the index time is large, it is conversely considered that neutrophils are excessively activated (the sensitivity to the stimulant is reduced). That is, it is possible to multilaterally evaluate the neutrophil activity by evaluating the index time and the peak area in combination.

What is claimed is:

1. A method of collecting data for determining whether or not a subject is suffering from a disease associated with oxidative stress due to excessively activated neutrophils, or whether or not the subject is in a condition associated with oxidative stress due to excessively activated neutrophils, the method comprising:
continuously measuring, for a predetermined amount of time, one or both of i) myeloperoxidase (MPO) activity and ii) superoxide production using a biological sample containing whole blood to which a neutrophil stimulant is added, by a value of fluorescence intensity or chemiluminescence intensity detected by one or both of a fluorescence detector and a luminescence measuring device, respectively,
wherein the MPO activity is measured by quantifying hypochlorous acid or hypochlorous acid halogen equivalents produced by MPO,
wherein the measurement is continued until a peak of the MPO activity or the superoxide production appears after the neutrophil stimulant is added to the biological sample,
wherein the data is a time from addition of the neutrophil stimulant to rise of a peak of the MPO activity or the superoxide production, and
wherein a point at which the peak of the MPO activity or the superoxide production rises is identified as a point at which a slope of the measurement value of fluorescence intensity or chemiluminescence intensity changes relative to the background level from a graph in which the measurement value is plotted with respect to the measurement time, the biological sample is a biological sample collected from the subject.

2. The method according to claim 1, further comprising:
before continuously measuring, measuring one or both of i) myeloperoxidase (MPO) activity and ii) superoxide production using a biological sample containing whole blood to which a neutrophil stimulant has not been added.

3. The method according to claim 1, further comprising:
deriving the time from addition of the neutrophil stimulant to rise of the peak of the MPO activity or the superoxide production based on a biological sample before treatment.

4. The method according to claim 2, further comprising:
deriving the time from addition of the neutrophil stimulant to rise of the peak of the MPO activity or the superoxide production based on the biological sample before treatment.

5. The method according to claim 1, wherein continuously measuring, for the predetermined amount of time, both of i) myeloperoxidase (MPO) activity and ii) superoxide production using the biological sample containing whole blood to which the neutrophil stimulant is added.

6. The method according to claim 2, wherein continuously measuring, for the predetermined amount of time, both of i) myeloperoxidase (MPO) activity and ii) superoxide production using the biological sample containing whole blood to which the neutrophil stimulant is added.

7. The method according to claim 3, wherein continuously measuring, for the predetermined amount of time, both of i) myeloperoxidase (MPO) activity and ii) superoxide production using the biological sample containing whole blood to which the neutrophil stimulant is added.

8. The method according to claim 4, wherein continuously measuring, for the predetermined amount of time, both of i) myeloperoxidase (MPO) activity and ii) superoxide production using the biological sample containing whole blood to which the neutrophil stimulant is added.

9. The method according to claim 1, wherein continuously measuring, for the predetermined amount of time, myeloperoxidase (MPO) activity using the biological sample containing whole blood to which a neutrophil stimulant is added.

10. The method according to claim 2, wherein continuously measuring, for the predetermined amount of time, myeloperoxidase (MPO) activity using the biological sample containing whole blood to which a neutrophil stimulant is added.

11. The method according to claim 3, wherein continuously measuring, for the predetermined amount of time, myeloperoxidase (MPO) activity using the biological sample containing whole blood to which the neutrophil stimulant is added.

12. The method according to claim 4, wherein continuously measuring, for the predetermined amount of time, myeloperoxidase (MPO) activity using the biological sample containing whole blood to which the neutrophil stimulant is added.

13. The method according to claim 1, wherein continuously measuring, for the predetermined amount of time, superoxide production using the biological sample containing whole blood to which the neutrophil stimulant is added.

14. The method according to claim 2, wherein continuously measuring, for the predetermined amount of time, superoxide production using the biological sample containing whole blood to which the neutrophil stimulant is added.

15. The method according to claim 3, wherein continuously measuring, for the predetermined amount of time, superoxide production using the biological sample containing whole blood to which the neutrophil stimulant is added.

16. The method according to claim 4, wherein continuously measuring, for the predetermined amount of time, superoxide production using the biological sample containing whole blood to which the neutrophil stimulant is added.
